(19) United States
(12) Patent Application Publication
(10) Pub. No.: US 2010/0249635 A1
(43) Pub. Date: Sep. 30, 2010

(54) HEARING SCREENING SYSTEM FOR A SUBJECT OR A PATIENT, AND A METHOD FOR HEARING SCREENING

(75) Inventor: Christoph Stefan Van Der Reijden, Best (NL)

Correspondence Address: THORNE & HALAJIAN, LLP, 111 WEST MAIN STREET, BAY SHORE, NY, 11706, US

(73) Assignee: CORDIAL MEDICAL EUROPE B.V., Eindhoven (NL)

(21) Appl. No.: 12/412,343

(22) Filed: Mar. 26, 2009

(51) Int. Cl. A61B 5/0478; A61B 5/00

(52) U.S. Cl. 600/544; 600/559

(57) ABSTRACT

An auditory stimulus device in a first aspect for applying an auditory stimulus to at least one ear of a subject; at least three electrodes for electrically connecting to at least three different positions on a head of the subject for measuring respective potential changes in response to the auditory stimulus; two galvanically isolated power supplies; two differential amplifiers arranged for receiving galvanically isolated supply voltages from the power supplies. In a second aspect the system comprises one (or more) electroencephalographic (EEG) channels with one differential amplifier, wherein the differential amplifier is arranged for amplifying a potential difference between the pair of electrodes for obtaining an amplified EEG signal on an output of the differential amplifier, wherein the differential amplifier is configured for first amplifying the potential difference with a total differential gain and subsequently taking a difference between respective output potentials of the amplified potential difference.

### FIELD OF THE PRESENT SYSTEM

The present system relates to a hearing screening system for a subject or patient, the system including: i) auditory stimulus device for applying an auditory stimulus to at least one ear of the subject or patient; and ii) two or more electrodes, such as at least three electrodes, for electrically connecting to different positions on a head of the subject or patient for measuring respective potential changes in response to the auditory stimulus. The present system further relates to a hearing screening method for a subject or patient.

### BACKGROUND OF THE PRESENT SYSTEM

Hearing is an essential feature to enable communication and to enjoy music. The ability to hear is also essential to develop language and to understand speech. Although it is not until the age of four years that a child is able to speak in grammatically correct sentences, auditory perception skills undergo significant development before the age of six months (Yoshinaga-Itano et al., “Language of earley—and later—identified children with hearing loss”, Pediatrics, 1998, v102, p1161-1171). This has resulted in the introduction of universal neonatal hearing screening programs in a number of countries. Two types of hearing screeners are used in these neonatal hearing screening programs.

A first type of hearing screener uses a probe that is put into the outer ear canal. This probe contains a loudspeaker and a microphone. The microphone listens to emissions of sound from the cochlea in response to a sound that is delivered to that ear by the loudspeaker. This is called oto-acoustic emissions (OAE). This technique is cheap, but has a limited sensitivity in detecting children with a possible hearing loss: A pass means that the cochlea is OK, but the integrity of the neural pathway after the cochlea leading to the auditory cortex is not tested. This is a big disadvantage of the OAE.

The second type of hearing screener tests the whole pathway (i.e. the acoustic and the neurological pathway): A sound stimulus (usually clicks) is delivered to the outer ear canal by a transducer (headphone, insert ear phone) and electrodes on the human head record the responses from the brainstem that are in synchrony with the stimulus. This method is called the Automated Auditory Brainstem Response (AABR). This method is the gold standard of hearing screening (J. W. Hall, New Handbook of Auditory Evoked Responses. 2007).

U.S. Pat. No. 5,230,344, the contents of which is incorporated by reference in its entirety, discloses an evoked potential processing system which includes, in one embodiment, a spectral averaging method. Time based, digital pre-stimulus and post-stimulus electroencephalographic (EEG) signal streams are obtained and are converted into frequency spectrum signals. A differential spectrum is obtained. The differential spectrums from a plurality of sweeps are summed. The summed differential spectrum is then converted into a time based signal stream which contains the evoked potential (EP) signal therein. The EP signal can also be obtained utilizing a two-dimensional filter. Pre- and post-stimulus EEG signal streams for a sub-group of stimuli, wherein each stimulus in a group has the same intensity or frequency, are filtered by conventional averaging or spectral differential averaging. The time based, filtered, post-stimulus EEG signal streams are placed in an array and the array is then filtered by a two-dimensional Fast Fourier Transform (FFT) filter. The array is then filtered by a mask and the masked array is then transformed into a time based format by an inverse FFT. The adaptive averaging technique utilizes a computational formula which computes an estimated running signal to noise ratio. When the difference between the pre-stimulus running SNR and the post-stimulus running SNR is less than a predetermined threshold, further stimulation and acquisition of EEG signals stops. Hence, the post-acquisition processing of the EEG signals is limited to that number of EEG sweeps. The electrode wire configuration uses a cross wiring scheme wherein the shield of a particular wire is connected to the other electrode wire to eliminate artifacts in the respective electrode wire.

While U.S. Pat. No. 5,230,344 shows an electrode wire configuration to eliminate or reduce noise or artifacts in the electrode pick up wiring. U.S. Pat. No. 5,099,856, the contents of which is incorporated by reference in its entirety, shows to additionally connect at least one of the electrodes to the power supply to reduce common mode noise and artifacts. However, it has been found by the inventors of the present system, that these configurations surprisingly reduce common mode rejection and thereby, increases noise and artifacts.

The next paragraphs describe different hearing screeners, based on the AABR technique. Most of them have the additional functionality of diagnosing hearing thresholds i.e. determine hearing thresholds (in dB) as a function of sound frequency (e.g. 0.5, 1, 2 and 4 kHz) also called an audiogram.

Intelligent Hearing Systems (IHS, Miami, Fla., USA, information available at intelligenthearingsystems.com) sells the “SmartScreener”. This product is a hearing screener with the option of hearing diagnostics. This product uses a personal computer or laptop to run software for hearing screening or hearing diagnostics. A universal smart box producing the auditory stimuli is connected to this personal computer or laptop. A second box containing at least two amplifiers is also connected to this personal computer or laptop. In a one-channel EEG recording configuration, at least three electrodes are connected to the amplifier-box by electrical wires. In a two-channel EEG recording configuration, at least four electrodes are connected to the amplifier-box (see their smart notes on their website about the ABR Screening using the Smart Screener). Connecting three electrodes to the skin in a one-channel EEG recording configuration increases preparation time. Connecting four electrodes in a dual-channel configuration to the skin increases even more the preparation time. Furthermore an increased number of electrodes increases the risk of bad electrode contact to the skin of the subject during hearing screening which increases testing time.

Interacoustics (Assens, Denmark, information available at interacoustics.dk) sells the ABRIS. This product is a hearing screener with the option of hearing diagnostics. The software of this product is based on scientific research of the German researchers: E. Stürzebecher and M. Cebulla. This product uses a personal computer or laptop to run software for hearing screening or hearing diagnostics. A box called the “Eclipse Platform” utilized to produce the auditory stimuli is connected to this personal computer or laptop. A second box containing at least two amplifiers is also connected to this personal computer or laptop. In a one-channel EEG recording configuration, at least three electrodes are connected to the amplifier-box by electrical wires. In a two-channel EEG recording configuration, at least four electrodes are connected to the amplifier-box (the Eclipse Operating Manual is available at interacoustics.com/com_en/Pages/Product/Abr/_index.htm?prodid=4774#). Connecting three electrodes to the skin in a one-channel EEG recording configuration increases preparation time. Connecting four electrodes in a dual-channel configuration to the skin increases even more the preparation time. Furthermore an increased number of electrodes increases the risk of bad electrode contact to the skin of the subject during hearing screening which increases testing time.

Maico-Diagnostic GmbH (Berlin, Germany, information available at maico-diagnostic.com) sells the Beraphone. This product is a hearing screener with the option of hearing diagnostics. The software is based on scientific research of the German researchers: E. Stürzebecher and M. Cebulla. The product comprises a unit, the Beraphone, which is placed on one of the ears. It produces sounds and a one-channel EEG is amplified. In this unit, three reusable electrodes are integrated to pick up the electrophysiological signals. This unit is connected to a personal computer or laptop on which software runs to evaluate the amplified one-channel EEG for responses to the auditory stimuli.

WO03/032811A2, the contents of which is incorporated by reference in its entirety, shows an apparatus and method for evaluation of hearing loss. The apparatus and method use evoked auditory brainstem responses (ABR) to determine if the subject is able to hear repeatedly administered click stimuli. In order to optimize evaluation, the apparatus uses normative data that is age dependent to weight the auditory responses, and to compensate for different or changing noise conditions. However, as such, the apparatus is complex to use.

A problem with the known hearing screening system is that it is not reliable enough in all circumstances.

### SUMMARY OF THE PRESENT SYSTEM

It is an object of the present system to overcome disadvantages and/or make improvements in the prior art.

In a first aspect, the present system relates to a hearing screening system for a subject or patient, wherein the system includes:

auditory stimulus device for applying an auditory stimulus to at least one ear of the subject or patient;

at least three electrodes for electrically connecting to at least three different positions on a head of the subject or patient for measuring respective potential changes in response to the auditory stimulus;

a first power supply having first supply terminals for supplying a first supply voltage;

a second power supply having second supply terminals for supplying a second supply voltage;

a first differential amplifier being arranged for receiving the first supply voltage, the first differential amplifier having a first pair of inputs coupled to a first pair of electrodes selected from the at least three electrodes, the first differential amplifier being arranged for amplifying a first potential difference between the first pair selected from the electrodes for obtaining a first EEG signal on a first output of the first differential amplifier, and

a second differential amplifier being arranged for receiving the second supply voltage, the second differential amplifier having a second pair of inputs coupled to a second pair of electrodes that is different than the first pair of electrodes and that is selected from the at least three electrodes, the second differential amplifier being arranged for amplifying a second potential difference between the second pair selected from the electrodes for obtaining a second EEG signal on a second output of the second differential amplifier;

wherein the first supply voltage and the second supply voltage are galvanically isolated from each other

Features of the hearing screening system in accordance with the present system will be explained hereinafter. The first differential amplifier is configured for amplifying a first potential difference between the first pair of electrodes for obtaining a first analog EEG signal. The second differential amplifier is configured for amplifying a second potential difference between the second pair of electrodes for obtaining a second analog EEG signal. Thus, two EEG channels can be measured with the two differential amplifiers with their respective electrodes, simultaneously. A feature of the present system is the provision of galvanically isolated power supplies for supplying the respective supply voltages for the differential amplifiers. The galvanic isolation between the power supply voltages effectively reduces the noise generated by one electrode pair/differential amplifier that is transferred to the other electrode pair/differential amplifier, and vice versa. Less noise results in a better signal to noise ratio, and thus a more reliable hearing screening system, because the likelihood of undetected responses in the EEG is reduced, in particular in environments with a lot of RF radiation (GSM, UMTS, WIFI, DECT, etc).

In an embodiment of the hearing screening system in accordance with the present system at least one of the first supply terminals is coupled to a first specific one of the electrodes, and at least one of the second supply terminals is coupled to a second specific one of the electrodes, wherein the second specific one is different from the first specific one. Due to the coupling between respective supply terminals of the respective amplifiers and the respective electrodes, the amplifier potentials are now electrically related to at least one of the electrodes carrying a subject or patient's body potential (this is also being referred to as potential balancing). As a result, the signals to be measured are more likely to fall within the measurement range of the amplifiers, which renders the system more tolerant to varying operating conditions, such as noise. A feature in this embodiment uses different electrodes for connecting to the respective one of the first and second supply terminals respectively. The inventor has found that the combination of this feature with the earlier described galvanic isolation feature opens up the possibility of measuring two EEG channels with only three electrodes. A problem with the known hearing screening system is that it is not suitable for producing two independent EEG channels without providing a 4^{th} electrode acting as reference electrode to the power supply.

In an embodiment of the hearing screening system in accordance with the present system the first supply terminals further comprise a first intermediate supply terminal for supplying a first intermediate supply potential that is located between respective potentials of the first supply voltage, and the second supply terminals further comprise a second intermediate supply terminal for supplying a second intermediate supply potential that is located between respective potentials of the second supply voltage. In this embodiment the first intermediate supply terminal is coupled to the first specific one of the electrodes and the second intermediate supply terminal is coupled to the second specific one of the electrodes. Coupling of the respective electrodes to an intermediate supply potential renders it easier to make the differential amplifier operate within its operation limits (input voltages are within supply potential range). In a first variant this intermediate level is fed to the differential amplifier, where it is used to define certain internal potentials. In a second variant the differential amplifier creates the intermediate level (i.e. ground) itself, for example by means of a voltage divider circuit between the supply terminals. In that case it is not required to provide such level to the differential amplifier via a terminal. What is important in the present system is that the respective reference electrodes are coupled to at least one of the power supply terminals for coupling the signal of the respective reference electrode thereto. In that case a common-mode rejection is achieved, which results in a better signal-to-noise ratio, and thereby a more accurate hearing screening system.

In an embodiment of the hearing screening system in accordance with the present system the at least three electrodes comprise a first electrode, a second electrode and a third electrode, respectively. In this embodiment the first pair of inputs is coupled to the first electrode and the third electrode, and the second pair of inputs is coupled to the second electrode and the third electrode, respectively. This embodiment constitutes an advantageous 3-electrode configuration, which results in two independent EEG channels, without requiring a separate (fourth) reference electrode to the power supplies. In this 3-electrode configuration in accordance with the present system one of the respective electrodes is shared between the differential amplifiers. This option is rendered possible by the combination of features of the previously discussed embodiments of the present system. Using only three electrodes saves costs over prior systems. Furthermore, the system becomes easier to implement, since it is relatively easier to position the electrodes, such as on a headset for a subject or patient, which is one of the advantageous embodiments discussed later in the description.

In an embodiment of the hearing screening system in accordance with the present system the first specific one of the electrodes that is coupled to the at least one of the first supply terminals, is the second electrode, and the second specific one of the electrodes that is coupled to the at least one of the second supply terminals, is the first electrode. The cross-coupling in this 3-electrode configuration is carefully chosen. Experiments have shown that this way of cross-coupling (supply of first amplifier coupled to the second electrode and supply of second amplifier coupled to first electrode) of the reference electrodes results in much less noise on the EEG channels.

In an embodiment of the hearing screening system in accordance with the present system each respective one of the first pair of inputs is provided with a first coupling for simultaneous DC-decoupling from and AC-coupling to a respective one of the first pair of electrodes. Furthermore, in this embodiment each respective one of the second pair of inputs is provided with a second coupling for simultaneous DC-decoupling from and AC-coupling to a respective one of the second pair of electrodes. The DC-decoupling on each input of the differential amplifier has the advantage that it enables the amplifier gain to be designed to be much larger without the amplifier having its output clamped to the supply voltages. In this embodiment, the inventor has realized that this technical measure is advantageous in a dedicated EEG pre-amplifier stage.

In a first variant of last mentioned embodiment of the hearing screening system in accordance with the present system, the respective coupling may include a respective coupling capacitor per input of the respective differential amplifiers, each respective coupling capacitor being connected between a respective electrode and a respective input. Such coupling capacitors can be easily integrated into the circuitry of which the differential amplifiers are part of.

In an embodiment of the hearing screening system in accordance with the present system the differential amplifiers are configured for a total differential gain between 1000 and 200000. In another embodiment of the hearing screening system in accordance with the present system the differential amplifiers are configured for a total differential gain between 5000 and 40000. In yet another embodiment of the hearing screening system in accordance with the present system the differential amplifiers are configured for a total differential gain between 10000 and 20000. These embodiments may be advantageous in combination with the embodiment in which DC decoupling is applied.

In an embodiment of the hearing screening system in accordance with the present system the differential amplifiers are configured for first amplifying the respective potential differences with the total differential gain and subsequently taking a difference between respective output potentials of the amplified potential differences. The taking of the difference is advantageously done in a last amplification stage of the differential amplifiers. In this embodiment a better common-mode rejection is achieved because of the larger gain prior to subtraction of the amplified differential signals in the last amplification stage within the differential amplifiers. This embodiment is advantageous because of the fact that a very good signal-to-noise ratio can be achieved. In the prior art pre-amplifiers in medical systems are always designed such that they are suitable for amplifying all kinds of bio-potentials (ECG, EEG, etc). Because of this requirement the pre-amplifiers are always designed such they first amplify the signal with a first factor which is kept small, and subsequently they take a difference between respective outputs of the pre-amplifier, where after the difference is further amplified with a second factor. It is the inventor who realized that such configuration is very disadvantageous for EEG signal amplification. The inventor realized that the difference should be taken at the very end of the pre-amplifier stage when the signal is amplified with the total differential gain of the pre-amplifier stage. A higher signal-to-noise ratio results in a more accurate hearing screening system because the EEG response to the hearing stimuli will be more profound and the risk of missing such response is reduced. It must be noted that the pre-amplifier part of this embodiment of the present system is also applicable to single-channel EEG hearing screening systems.

In an embodiment of the hearing screening system in accordance with the present system the differential amplifiers each comprise multiple stages to obtain the total differential gain. Designing the differential amplifiers with multiple stages renders the design of the differential amplifiers easier in terms of satisfying all electrical requirements (low noise, differential gain, common-mode rejection factor, harmonic distortion, etc).

In an embodiment of the hearing screening system in accordance with the present system the differential amplifiers each comprise a respective first differential stage comprising a junction-field-effect-transistor-based current mirror that is driven by a constant-current source. The inventor has discovered that the JFET-based current mirror in combination with the constant-current source provides for a very good common-mode rejection. JFET's are advantageous because of their low-noise figures (compared to bipolar transistors for example). Another advantage is their very low gate currents and high input impedance.

In an embodiment of the hearing screening system in accordance with the present system the junction-field-effect-transistor based current mirror is configured for a first gain between 20 and 100. In another embodiment of the hearing screening system in accordance with the present system the junction-field-effect-transistor based current mirror is configured for a first gain between 30 and 50. In an embodiment of the present system the current mirror is designed with a common-mode rejection below 1. In another embodiment of the present system the current mirror is designed with a common-mode rejection below 0.5. In yet another embodiment of the present system the current mirror is designed with a common-mode rejection below 0.1.

In an embodiment of the hearing screening system in accordance with the present system the differential amplifiers each further comprise a respective second stage cascaded to the first stage, wherein the second stage is configured for a second gain between 50 and 2000. In another embodiment of the present system the second stage is configured for a second gain between 100 and 1000. In yet another embodiment of the present system the second stage is configured for a second gain between 300 and 500.

In an embodiment of the hearing screening system in accordance with the present system the differential amplifiers each further comprise a respective third stage cascaded to the second stage, wherein the third stage is configured for taking a difference between respective outputs of the second stage.

In an embodiment of the hearing screening system in accordance with the present system the differential amplifiers each further comprise a respective fourth stage which is configured for DC leveling of the respective outputs of the differential amplifiers.

In an embodiment of the hearing screening system in accordance with the present system the fourth stage also provides the function of taking the difference.

An embodiment of the hearing screening system in accordance with the present system further comprises two isolation amplifiers and a third power supply. Furthermore, in this embodiment each respective one of the differential amplifiers is coupled to a respective one of the isolation amplifiers, wherein the isolation amplifiers each comprise a galvanic barrier between a respective input side and a respective output side thereof, wherein the respective output sides are each coupled to the third power supply for setting a DC level of respective outputs of the isolation amplifiers to obtain a first output channel and a second output channel respectively. This embodiment of the system conveniently enables the coupling of the two EEG channels to a single processor unit.

In one embodiment of the present system, the system is integrated into a head-set for mounting on the head of a subject or patient. Integration of the system into a head-set is advantageous in the hearing screening application. The head-set may be put on the head of the subject or patient and connected to a processor unit via cables. The subject or patient can be lying down or have any other position/orientation. The head-set in this embodiment is referred to herein as brainstem recorder.

In an embodiment of the hearing screening system in accordance with the present system the head-set is designed such that, in operational use, a position of the first electrode coincides with the right-mastoid position, a position of the second electrode coincides with the left-mastoid position, and a position of the third electrode coincides with the Cz-position, respectively. These electrode positions provide for a very good signal-to-noise ratio of the auditory evoked response for most of the subjects or patients. The left mastoid is the location on the bone behind the left ear, the right mastoid is the location on the bone behind the right ear, and Cz (or vertex) is the location on top of the head exactly between the left ear and the right ear. Thus the locations selected in this embodiment make the integration of the system into the head-set much easier. A further advantage of these electrode positions is that the contact between the electrodes and the skin of the subject or patient can be established by mounting the headset on the head of the subject or patient from a backside of the head.

In an embodiment of the hearing screening system in accordance with the present system the head-set comprises ear-caps for receiving the ears of the subject or patient and for guiding sound from the auditory stimulus device (SPL, SPR) to the ears. In an embodiment the electrodes are located close to the ear caps, but not integrated into them. This has the advantage that the electrodes can be removed or replaced independently from the ear caps. In an alternative embodiment the electrodes are integrated into the ear caps.

In an embodiment of the hearing screening system in accordance with the present system the third electrode at the Cz-position is provided with an adjustment device to make the head-set fit on various head sizes. This embodiment is advantageous, because it makes the head-set fit onto a larger number of subjects or patients, i.e. it is more tolerant to head size variations. Furthermore, the third electrode in combination with the adjustment device can be used to prevent the head-set from gliding downwards when mounted on the head of the subject or patient (this would make the head-phones be dislocated with respect to the ears). Also this may secure the electrical connection between the third electrode and the inputs of the differential amplifiers in this embodiment.

In an embodiment of the hearing screening system in accordance with the present system the electrodes are durable electrodes. In prior systems, electrodes typically are just thrown away after a single use. The use of durable electrodes prevents such waste which is advantageous for the environment.

In an embodiment of the hearing screening system in accordance with the present system the electrodes comprise stainless steel, silver chloride, or sintered silver chloride.

In an embodiment of the hearing screening system in accordance with the present system the device for applying the auditory stimulus are configured for applying the auditory stimulus on both ears simultaneously at different repetition rates. The auditory stimulus can be a click, a tone-pip or any combination of frequencies. The auditory stimulus can also be a combination of continuous frequencies that are independently modulated in amplitude and frequency with independent modulation frequencies. This embodiment is advantageous because both ears can be screened at the same time. The different stimulus rates that are applied left and right will occur in the frequency spectrum of the measured EEG channels also and can be distinguished from each other when using the proper data processing techniques.

An embodiment of the hearing screening system in accordance with the present system comprises a processor unit connected to the auditory stimulus device for controlling the application of the auditory stimulus, the processor unit being further configured for receiving, collecting, and processing data from the differential amplifier to obtain quantitative data about the hearing ability of the subject or patient. This embodiment of the hearing screening system provides a complete solution to hearing screening and may be sold as such.

In an embodiment of the hearing screening system in accordance with the present system the processor unit is arranged for calculating a third EEG channel by subtracting the first channel from the second channel. For some subjects or patients it may turn out that this third channel that is derived from the first channel and the second channel provides for the highest signal-to-noise ratio and thus the highest accuracy of the hearing screening system.

In a second aspect, the present system relates to a hearing screening system for a subject or patient, the system comprising:

auditory stimulus device for applying an auditory stimulus to at least one ear of the subject or patient;

at least two electrodes for electrically connecting to at least two different positions on a head of the subject or patient for measuring respective potential changes in response to the auditory stimulus;

a differential amplifier having a pair of inputs coupled to a pair selected from the electrodes, the differential amplifier being arranged for amplifying a potential difference between the pair of electrodes for obtaining an amplified EEG signal on an output of the differential amplifier, wherein the differential amplifier is configured for a total differential gain between 1000 and 200000, and wherein the differential amplifier is configured for first amplifying the potential difference with the total differential gain and subsequently taking a difference between respective output potentials of the amplified potential difference. The advantages and effects of this single-channel embodiment and all further embodiments discussed hereinafter follow that of the corresponding 2-channel embodiments of the hearing screening system in accordance with the first aspect of the present system. It must be noted that this particular aspect of the present system is applicable to a hearing screen system using any positive number of channels (1 and higher).

With the system in accordance with the second aspect very good signal-to-noise ratio can be achieved. Prior art pre-amplifiers in medical systems are always designed such that they are suitable for amplifying all kinds of bio-potentials (ECG, EEG, etc). Due to this requirement in prior systems the pre-amplifier is always designed such it first amplifies the signal with a first factor which is kept small, and subsequently a difference is taken between respective outputs of the pre-amplifier, where after the difference is further amplified with a second factor. It is the inventor who realized that such configuration is very disadvantageous for EEG signal amplification even in single channel EEG systems. The inventor realized that the difference should be taken at the very end of the pre-amplifier stage when the signal is amplified with the total differential gain of the pre-amplifier stage. A higher signal-to-noise ratio results in a more accurate hearing screening system because the EEG response to the hearing stimuli will be more profound and the risk of missing such response is reduced.

In a further embodiment of the hearing screening system in accordance with the present system the total differential gain lies between 5000 and 40000. In a further embodiment of the hearing screening system in accordance with the present system the total differential gain lies between 10000 and 20000.

In an embodiment of the hearing screening system in accordance with the present system the differential amplifier comprises multiple stages to obtain the total differential gain.

In an embodiment of the hearing screening system in accordance with the present system the differential amplifier includes a respective first differential stage comprising a junction-field-effect-transistor-based current mirror that is driven by a constant-current source.

In an embodiment of the hearing screening system in accordance with the present system the junction-field-effect-transistor based current mirror is configured for a first gain between 20 and 100. In a further embodiment of the hearing screening system in accordance with the present system the current mirror is configured for a first gain between 30 and 50. In an embodiment of the present system the current mirror is designed with a common-mode rejection below 1. In another embodiment of the present system the current mirror is designed with a common-mode rejection below 0.5. In yet another embodiment of the present system the current mirror is designed with a common-mode rejection below 0.1.

In an embodiment of the hearing screening system in accordance with the present system the differential amplifiers each further include a respective second stage cascaded to the first stage, wherein the second stage is configured for a second gain between 50 and 2000. In another embodiment of the present system the second stage is configured for a second gain between 100 and 1000. In yet another embodiment of the present system the second stage is configured for a second gain between 300 and 500.

In an embodiment of the hearing screening system in accordance with the present system the differential amplifiers each further comprise a respective third stage cascaded to the second stage, wherein the third stage is configured for taking a difference between respective outputs of the second stage.

In an embodiment of the hearing screening system in accordance with the present system the differential amplifiers each further include a respective fourth stage which is configured for DC leveling of the respective outputs of the differential amplifiers.

In an embodiment of the hearing screening system in accordance with the present system the fourth stage also provides the function of taking the difference.

In a third aspect, the present system relates to a hearing screening method for a subject or patient, the method comprising:

providing at least two electrodes on at least two different positions on a head of the subject or patient;

applying an auditory stimulus to at least one ear of the subject or patient;

measuring potential changes in response to the auditory stimulus with the at least two electrodes, and

amplifying a potential difference between the electrodes with a total differential gain between 1000 and 200000 for obtaining an amplified EEG signal, and

taking a difference between respective output potentials of the amplified potential difference.

The advantages and effects of this method follow that of the corresponding embodiment of the hearing screening system in accordance with the second aspect of the present system. The method has embodiments which follow that of corresponding embodiments of the hearing screening system.

It must be noted that, in accordance with the present system, the embodiments mentioned above can be combined, unless it is explicitly mentioned that such combination is not possible or not useful.

These and other aspects of the present system are apparent from and will be elucidated with reference to the embodiments described hereinafter.

### BRIEF DESCRIPTION OF THE DRAWINGS

In the drawings:

FIG. 1 shows a hearing screening system in accordance with a first embodiment of the present system;

FIG. 2a shows part of a pre-amplifier stage in the hearing screening system of FIG. 1;

FIG. 2b shows a first variant of the pre-amplifier stage of FIG. 2a in accordance with a second embodiment of the present system;

FIG. 2c shows a second variant of the pre-amplifier stage of FIG. 2a in accordance with a third embodiment of the present system;

FIG. 3 shows an embodiment of the differential amplifiers in the hearing screening system of the present system;

FIG. 4a shows an illustration of a head-set comprising the hearing screening system of FIG. 1 and a processor unit;

FIG. 4b shows an illustration of a front-side of the head-set of FIG. 4a, and

FIG. 4c shows an illustration of a rear-side of the head-set of FIG. 4a.

### LIST OF REFERENCE NUMERALS

10 head of patient or subject
50 adjustment device for third electrode
AMP integrated amplifier
HB flexible head-band
SPL speaker left
SPR speaker right
ML microphone left
MR microphone right
EC ear-caps
H hinges
EL1 first electrode
EL2 second electrode
EL3 third electrode
PS1 first power supply (galvanically isolated from second and third power supplies)
PS2 second power supply (galvanically isolated from first and third power supplies)
PS3 third power supply (galvanically isolated from first and second power supplies)
DA1 first differential amplifier (part of pre-amplifier stage, channel 1)
DA2 second differential amplifier (part of pre-amplifier stage, channel 2)
GND1 intermediate supply voltage of first power supply (i.e. ground or 0V)
VDD1 first supply voltage (potential) of first power supply (i.e. +15V)
VSS1 second supply voltage (potential) of first power supply (i.e. −15V)
GND2 intermediate supply voltage of second power supply (i.e. ground or 0V)
VDD2 first supply voltage (potential) of second power supply (i.e. +15V)
VSS2 second supply voltage (potential) of second power supply (i.e. −15V)
GND3 intermediate supply voltage of third power supply (i.e. ground or 0V)
VDD3 first supply voltage (potential) of third power supply (i.e. +15V)
VSS3 second supply voltage (potential) of third power supply (i.e. −15V)
C11 first coupling capacitor on first input of first differential amplifier
C12 second coupling capacitor on second input of first differential amplifier
C21 first coupling capacitor on first input of second differential amplifier
C22 second coupling capacitor on second input of second differential amplifier
IA1 first isolation amplifier (channel 1)
IA2 second isolation amplifier (channel 2)
IS input side of isolation amplifiers
OS output side of isolation amplifiers
GB galvanic barrier of isolation amplifiers
CH1 first channel
CH2 second channel
R1 first resistor (determines with R2 the differential gain of JFET current mirror and determines with ZCCS the common-mode gain of JFET current mirror)
R2 second resistor (determines with R1 the differential gain of JFET current mirror)
R3 third resistor (determines gain of AD8221)
ZCCS impedance of constant current source (CCS) (determines with R1 the common-mode gain of JFET current mirror)
BR balancing resistor
JF1 first junction FET
JF2 second junction FET
V_{OUT} output voltage of differential amplifier
EIA precision instrumentation amplifier
AD8221 precision instrumentation amplifier standard component
LSK389 single package dual-JFET standard component

### DETAILED DESCRIPTION OF THE PREFERRED EMBODIMENT

The following are descriptions of illustrative embodiments that when taken in conjunction with the following drawings will demonstrate the above noted features and advantages, as well as further ones. In the following description, for purposes of explanation rather than limitation, illustrative details are set forth such as architecture, interfaces, techniques, element attributes, etc. However, it will be apparent to those of ordinary skill in the art that other embodiments that depart from these details would still be understood to be within the scope of the appended claims. Moreover, for the purpose of clarity, detailed descriptions of well known devices, circuits, tools, techniques and methods are omitted so as not to obscure the description of the present system. It should be expressly understood that the drawings are included for illustrative purposes and do not represent the scope of the present system.

The present system aims at providing a hearing screening system which is able to operate under a wider range of different operating conditions, in particular in environments in which there is a lot of RF radiation. The present system provides such solution in different manners. First, the present system provides a 2-channel (or more channels) EEG hearing screening system in which each channel has its own differential amplifier, wherein the respective differential amplifiers (located in a pre-amplifier stage) receive their respective supply voltages by two independent galvanically isolated power supplies. The inventor has discovered that galvanic separation of the respective power supplies increases the reliability of the hearing screening system to a great extent. Furthermore such measure opens up a lot of new opportunities which are exploited in the advantageous embodiments.

Second, the present system provides a 1-channel (or more channels) EEG hearing screening system in which the differential amplifiers in the preamplifier stage are designed such that they amplify the respective signals with a total differential gain between 1000 and 200000 first, where after a difference is taken between the respective differential outputs in the respective differential amplifiers. In the present system no significant further amplification is carried out after taking the difference. By doing so it is achieved that the common-mode rejection of the EEG signals is maximized, which provides a high signal-to-noise ratio on the outputs of the differential amplifiers. The higher signal-to-noise ratio on the output leads to a more reliable hearing screening system (less risk of missing brainstem responses). A further advantage of the present system is its extremely low power consumption of about 30 mW (1 mA×30 V) per EEG channel. This facilitates application of the present system in areas without a mains power supply but with solar power or other means of renewable energy present.

In order to facilitate the discussion of the detailed embodiments a few expressions are defined hereinafter.

Throughout this description the term “galvanic isolation” should be interpreted such that there is no galvanic connection between the involved nodes which are galvanically isolated, i.e. that respective potentials are not related to each other.

For purposes of simplifying a description of the present system, the terms “operatively coupled”, “coupled” and formatives thereof as utilized herein refer to a connection between devices and/or portions thereof that enables operation in accordance with the present system.

Potentially, all newborns worldwide (130 min/year) should be tested for a possible hearing loss by a hearing screener within the first four months after birth. Of course, only if there is a follow-up program, hearing screening makes sense. A hearing screener is intended to be used by e.g. a midwife or a nurse. Only a short training is needed to screen the ears of newborns correctly, no interpretation of test-results is needed: The outcome may be simply a pass or a refer. A pass means that hearing is OK. A refer means that additional hearing tests are needed. The nurse explains to the parents the screening procedure. After that, the nurse places the audio-transducers and the electrodes at the right positions of the child's head. The hearing is screened within about 2-10 minutes depending on state of sleep/rest/restlessness: sleep or rest reduces the test-time and improves a reliable outcome of the test significantly. After that, the audio-transducers and the electrodes are removed from the child's head and the nurse moves on to the next child. After a full day of hearing screening, the nurse may connect the screening device to a computer that is connected to a network such as the Internet to upload the screening outcomes of that day. Other nurses may also upload their screening results. In this way, all screening results may be transferred to one central computer. A software program may calculate the hearing screening statistics of that region/land or state (e.g. number of baby's tested, number of pass/refer).

Throughout this description the term “receiving the supply voltage” should be interpreted as receiving at least a first supply potential and a second supply potential different from the first potential. Alternatively, it may be interpreted as receiving a third supply potential different from the first supply potential and the second supply potential (for example a ground). The context of the description will make clear which situation is meant.

Throughout this description the term “taking a difference” means that a difference is taken between respective output levels of the amplified differential signals in the differential amplifiers. Often this is a voltage, but this is not essential. A difference can be taken by means of a differential amplifier circuit having feedback resistors such that the amplification factor is around 1. It is not essential that the amplification factor is exactly one, although this is advantageous. There may still be a minor positive or negative amplification. The amplification factor may be in a range between 0.5 and 2 for example.

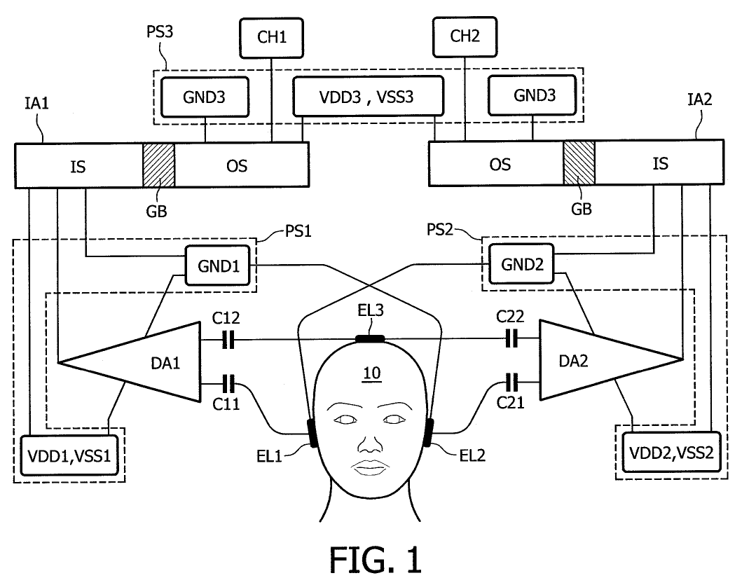

FIG. 1 shows a hearing screening system in accordance with a first embodiment of the present system. Notwithstanding the differences with the known systems, the part of the system as illustrated in this figure is also referred to as a brainstem recorder system or a hearing screener. FIG. 1 shows a head 10 of a patient or subject on which three electrodes is provided. A first electrode EL1 is provided at a location near the right ear (right mastoid), a second electrode EL2 is provided at a location near the left ear (left mastoid), and a third electrode EL3 is provided at a location on top of the head (Cz or vertex). Although these locations have been verified by the inventor as being advantageous in terms of signal-to-noise ratio of the evoked response to the auditory stimulus, the present system is not restricted to these electrode positions. The system in FIG. 1 constitutes a hearing screening system wherein a brainstem response is measured using two EEG channels CH1, CH2.

The first channel CH1 is measured as follows. A first differential amplifier DA1 is coupled to the first electrode EL1 and the third electrode EL3 and is arranged for amplifying the potential difference (first EEG-signal) between the first electrode EL1 and the third electrode EL3. In this embodiment the inputs of the respective amplifier DA1 are provided with coupling capacitors C11, C12. By doing so, the DC component of the voltage difference (which is a time varying quantity, i.e. a signal) is subtracted from the inputs. The inventor has realized that this allows the gain of the differential amplifier DA1 to be designed much larger, i.e. between 1000 and 200000 times, which is very beneficial for EEG signals, which are generally “drowned in noise”, i.e. the signals are very weak. It is not essential that decoupling capacitors are used on the input as a decoupling device, as long as a device or circuit is used which provides simultaneous DC-decoupling from and AC-coupling to the advantage of this embodiment is present.

The second channel CH2 is measured similarly. A second differential amplifier DA2 is coupled to the second electrode EL2 and to the third electrode EL3 and is arranged for amplifying the potential difference (second EEG-signal) between the second electrode EL2 and the third electrode EL3. In this embodiment the inputs of the respective amplifier DA2 are also provided with coupling capacitors C21, C22. By doing so, the DC component of the voltage difference is subtracted from the inputs.

The measurement of two EEG-channels in a brainstem recorder system or hearing screener system in accordance with the present system increases the reliability of the system. Nevertheless, the inventor has realized that better results can be achieved by using galvanically isolated power supplies for the respective differential amplifiers DA1, DA2. A first power supply PS1 is provided for supplying a first supply voltage VDD1, VSS1, GND1 to the first differential amplifier DA1. A second power supply PS2, which is galvanically isolated from the first power supply PS1, is provided for supplying a second supply voltage VDD2, VSS2, GND2 to the second differential amplifier DA2. The power supplies can be virtually any sort of power supply, but in an advantageous embodiment they comprise batteries. In this embodiment both power supplies are configured for respectively providing a first supply potential VDD1, VDD2, such as +15V, a second supply potential VSS1, VSS2, such as −15V, and an intermediate supply potential GND1, GND2, such as 0V (may be called ground, but this is an arbitrary choice). The supply voltages can be changed in accordance with the requirements of the circuit. In any case, the galvanic isolation between both channels reduces the noise generated by one channel which is induced in the other channel, and thereby increases the signal integrity of the system. This particular embodiment, however, goes further in improving the signal integrity.

A further improvement is obtained by coupling the intermediate supply potential GND1 of the first differential amplifier DA1 to the second electrode EL2, and by coupling the intermediate supply potential GND2 of the second differential amplifier DA2 to the first electrode EL1. In this way, the electrodes EL1, EL2 act as a corresponding input to the intermediate supply potential GND1, GND2. By doing so the intermediate supply potential GND1 of the first differential amplifier DA1 of the first channel CH1 moves along with the potential on the second electrode EL2, and the intermediate supply potential GND2 of the second differential amplifier DA2 of the second channel CH2 moves along with the potential on the first electrode EL1. This measure results in a clear common-mode rejection effect.

It must be noted that, instead, the respective ground levels could be connected to any other one of the electrodes. Nevertheless, such configuration suffers more from noise on the channels as the one illustrated in FIG. 1, i.e. the configuration in FIG. 1 is advantageous as experiments have shown that it provides very high signal integrity (noise reduction) on the channels. In these embodiments, the respective ground potentials are at least not connected to the same electrode as that would immediately couple the power supplies again. However, instead of coupling the ground potentials of the respective power supplies to one of the electrodes, also one of the other supply potentials VSS1, VSS2, VDD1, VDD2 could be taken (i.e. it is not essential to have a three potential power supply).

In the example of FIG. 1 both channels are effectively “brought together” (related to each other) by means of respective isolation amplifiers IA1, IA2. The isolation amplifiers each have a respective input side IS which each receive the respective supply potentials VSS1, VSS2, VDD1, VDD2, GND1, GND2 and respective output of the differential amplifiers. Further, the isolation amplifiers have a respective output side OS which is galvanically isolated from the respective input side IS by means of a galvanic barrier GB. Between the differential amplifiers DA1, DA2 and the isolation amplifiers IA1, IA2 filter circuitry may be added to improve the signal integrity.

Isolation amplifiers as such are well-known in the prior art. One of such known isolation amplifiers is the ISO122 from Burr-Brown Corporation. The respective output sides OS are both fed by a third power supply PS3, which is galvanically isolated from the first and second power supplies PS1, PS2. In this embodiment the third power supply is configured for providing a first supply potential VDD3, such as +15V, a second supply potential VSS3, such as −15V, and an intermediate supply potential GND3, such a 0V (may be called ground, but this an arbitrary choice). In accordance with an embodiment of the present system the two channels are brought together (coupled) at this point of the system. Alternatively, it may be done at another point in the flow. It is also possible that two individual processor units are coupled to the respective channels, and that the coupling is done thereafter.

It must be noted that the inventor is the first who provides a hearing screening system which only needs three electrodes to provide a dual-channel system. All prior solutions known so far need some kind of 4^{th} reference electrode to one of the supply voltages of the differential amplifier (a conductive wrist band or at least one more electrode). Less electrodes in accordance with the present system means less cost, less handling time of the system and improved reliability of electrode contact to the skin of the subject (easier to use, faster to apply to a patient or subject, good quality EEG during the whole recording session).

Nevertheless, the present system is not restricted to three-electrode configurations only; it may be carried out with four electrodes or more. For example, the electrode on the Cz-position may be doubled (and kept spaced apart).

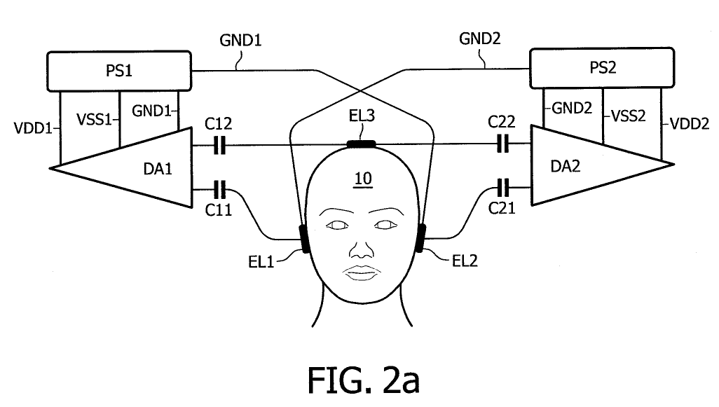

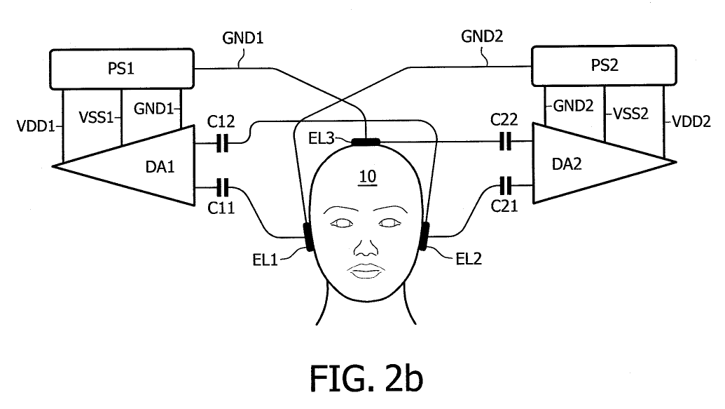

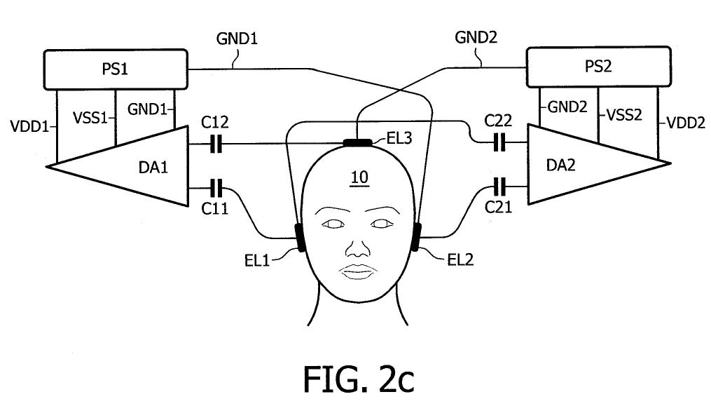

FIG. 2a shows part of a pre-amplifier stage in the hearing screening system of FIG. 1. This figure only constitutes a different representation for the corresponding part in FIG. 1, i.e. the respective galvanically isolated power supplies are now presented as clear individual blocks. FIG. 2b shows a first variant of the pre-amplifier stage of FIG. 2a in accordance with a second embodiment of the present system and FIG. 2c shows a second variant of the pre-amplifier stage of FIG. 2a in accordance with a third embodiment of the present system. When three electrodes are considered, mathematically three pairs of electrodes may be selected from them, i.e. three EEG channels can be measured. FIG. 2b and FIG. 2c illustrate the other two options which are available next to FIG. 2a (which is directed to a cross-coupled ground potential configuration applied to Left-mastoid, Right-mastoid, and Cz, respectively, neither of which being essential to the present system in the broadest sense).

The configuration in FIG. 2b provides a first channel which subtracts the Left-mastoid potential from the Right-mastoid potential. The second channel subtracts the Left-mastoid potential from the Cz potential (as in FIG. 2a). Now, the original first channel of FIG. 2a (i.e. a third channel) may be achieved by subtracting the second channel from the first channel (Cz minus Right-mastoid is obtained then).

The configuration in FIG. 2c provides a first channel which subtracts the Right-mastoid potential from the Cz potential (as in FIG. 2a). The second channel subtracts the Left-mastoid potential from the Right-mastoid potential. Now, the original second channel of FIG. 2a (i.e. a third channel) may be achieved by subtracting the second channel from the first channel (Cz minus Left-mastoid is obtained then).

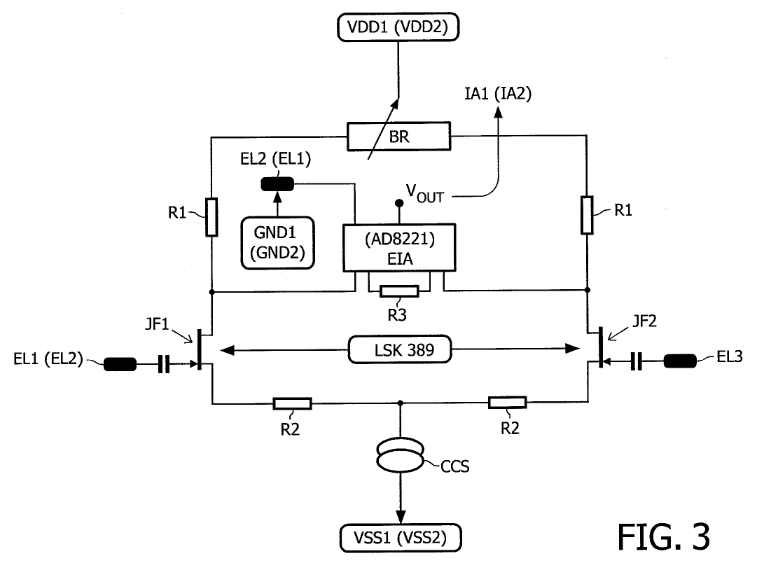

FIG. 3 shows an embodiment of the differential amplifiers in the hearing screening system of the present system. The differential amplifier in FIG. 3 is a multi-stage differential amplifier. A first stage is constituted by a junction-field-effect-transistor (JFET) based current mirror. The first stage comprises two JFET's JF1, JF2, first resistors R1, second (feedback) resistors R2, a constant-current source CSS, and a balancing resistor BR. The components are connected as illustrated in the drawing. Use of a current mirrors as such is known to the person skilled in the art.

In accordance with the present system, the current mirror is driven between the respective supply potentials VDD1, VDD2, VSS1, VSS2. FIG. 3 illustrates how the electrode potentials are applied to the inputs. In accordance with FIG. 1, on the first JF1 it is either the first electrode EL1 or the second electrode EL2, on the second JF2 it is the third electrode EL3 in both amplifiers. In different electrode configuration this can be different. In accordance with an embodiment of the present system, the JFET's shown in FIG. 3 may both be contained in a single package. For example, a package is available on the market as single package dual-JFET standard component LSK389 from Linear Integrated Systems Inc. More information on the LSK389 from Linear Integrated Systems Inc. is to be found in the datasheets, which are available at linearsystems.com. The data sheet is hereby incorporated by reference in its entirety. The inventor has realized that a first important step in the pre-amplifier is to suppress the common-mode signal on the inputs and that this is may be advantageously done using the JFET current mirror, for example including the constant-current source. With proper choosing of the first and second resistors R1, R2 and the impedance of the constant-current source CSS, R_{CCS}, a common-mode gain of about 1/10^{th} may be achieved ((R1)/R_{CCS} for example with R1=18 kΩ; R2=470 Ω, R_{CCS}=200 kΩ). At the same time the difference gain of about 38.3 is achieved (R1/R2). The constant-current source may be a temperature-stable JFET (U404) with a 200 Ω resistor between gate and source, for example (component is commercially available). In an illustrative embodiment, the first stage in FIG. 3 may be coupled with its output to a precision instrumentation amplifier EIA, which for example may be another standard component, i.e. the AD8221. More information on the AD8221 from Analog Devices Inc. is to be found in the datasheets, which are available at analog.com. The datasheet is hereby incorporated by reference in its entirety.

The gain of the instrumentation amplifier EIA is set by a third resistor R3, which is illustratively chosen to be 127 Ω in this embodiment. The gain of the instrumentation amplifier as shown in the datasheet is given by: G=1+(49.4 kΩ/R3)≈390. This brings the total differential gain of the differential amplifier stage to about 15000. It is not essential to have such gain. For EEG amplification it may be somewhere between 1000 and 200000. Nevertheless, what is important is that in this embodiment the total differential amplifier is designed such that first the signal is amplified with this factor, after which a difference is taken by a difference stage.

As is readily appreciated by a person of ordinary skill in the art, suitable resistors may be selected for achieving a desired gain in accordance with embodiments of the present system.

In FIG. 3, this difference is taken by the last stage in the instrumentation amplifier EIA, which in this embodiment also offers the function of DC leveling of the output signal Vout. Effectively, there are three stages before the stage taking the difference in this embodiment before the difference is taken (the JFET current-mirror and the first two stages in the instrumentation amplifier EIA). However, it is not essential to use three amplifier stages. Nevertheless, in accordance with the illustrative embodiment shown, the design of the circuit may be simplified. In accordance with the embodiment of the present system the signal is not substantially further amplified after the difference is taken as that would adversely affect the signal-to-noise ratio.

The precision instrumentation amplifier in FIG. 3 is also configured for receiving the respective supply potentials. For example, the instrumentation amplifier EIA may also receive a reference potential, such as GND2 or GND1, which as shown with reference to FIGS. 2a-2c, may be coupled to one of the electrodes (e.g., e.g., GND2 and/or EL2 when JF1 is coupled to EL1). For reasons of clarity these are not drawn. Furthermore, the differential amplifier in FIG. 3 may be provided with appropriate DC-level setting, which also is left out for clarity reasons.

As may be readily appreciated, the configuration shown in FIG. 3 is suited for a first aspect of the present system which provides a hearing screening system which only needs three electrodes to provide a dual-channel system. Further, the configuration shown in FIG. 3 is suited for a second aspect of the present system which provides a hearing screening system which utilizes at least two electrodes to provide at least a single-channel system wherein respective potential differences are first amplified with the total differential gain and subsequently a difference between respective output potentials of the amplified potential differences is provided. In the second aspect, the instrumentation amplifier EIA may also receive a reference potential without being coupled to one of the electrodes. The taking of the difference is advantageously done in a last amplification stage of the differential amplifiers with regard to both the first aspect and the second aspect of the present system in these embodiments.

In general, it must be noted that the inventor realized that a full-analog approach as in FIG. 3 provides much better results than the more digital solutions as known from the prior art. In the prior art the aim is to go to the digital domain as soon as possible, which, as the inventor has realized, is very bad for the signal to noise ratio. Also, the prior art systems are configured for measuring many different bio-potentials (EEG, ECG, etc). The inventor has realized that this choice for a general system poses a severe limitation to the common-mode-rejection-ratios (signal-to-noise ratio) which are achieved, and thereby the reliability of the hearing screening system is adversely affected as well. With the configuration of FIG. 3 the inventor has measured common-mode-rejection ratio over 140 dB, whereas the best performance known from the prior art is in the order of 120 dB.

The above description can also be formulated differently. One major source of noise in EEG recordings is common-mode noise i.e. equal signals on both inputs at the same time. A difference amplifier is designed to optimize the differential gain while reducing the common mode gain as much as possible. In prior art an optimized differential amplifier is called an instrumentation amplifier. An instrumentation amplifier with the classical three-opamp design is widely used to amplify small differential signals in an environment with large common-mode signals. Low harmonic distortion is achieved by using a moderate amplification of between 10 to 100 times. The first two opamps in an instrumentation amplifier, amplify the differential signal with a differential gain of between 10 to 100 times while the common-mode signal passes with a gain of one. A third opamp subtracts the common mode signal from the amplified differential signals. Unfortunately, in real-world applications, common-mode signals are never subtracted completely from the differential signals leaving a small amount of them in the output of the instrumentation amplifier. Further amplification of the instrumentation amplifiers output will not improve common-mode rejection because both differential signals and common-mode signals will be amplified equally. In the present system an embodiment is described wherein with a further improvement of the instrumentation amplifier. In the first amplification stage, using a current mirror and matched low-noise (0.9 nV/sqrt(Hz)) JFETs, the common-mode noise is reduced with a factor of 10 (e.g., gain is 0.1 and not 1 like in an instrumentation amplifier) while the differential gain may be about 30 times. The output of this first amplification stage is then followed by a second amplification stage, for example with about 390 times amplification, for example using an excellent instrumentation amplifier. So, in accordance with an embodiment of the present system common-mode signals are reduced in the first amplification stage, for example by 20 dB as compared to conventional instrumentation amplifiers.

In accordance with the present system, one or more electrical properties for the differential amplifiers may include:

low noise (<1 nV/sqrt(Hz));

low input bias-current through the electrodes to the JFETs (is fulfilled because the extremely low gate-current of the JFETs and because the DC-decoupling capacitors prevent bias currents to flow to the electrodes);

high common-mode rejection ratio (i.e. low common-mode gain and high differential gain), and

moderate differential gain in first amplification stage to prevent harmonic distortion.

The differential gain is determined by the ratio of R1 and R2. The common-mode gain is determined by the ratio of R1 and the impedance of the constant current source (CCS). Ideal constant current sources have an infinite impedance. In practice, constant current sources have finite, yet high, impedances of about 100 kΩ-500 kΩ.

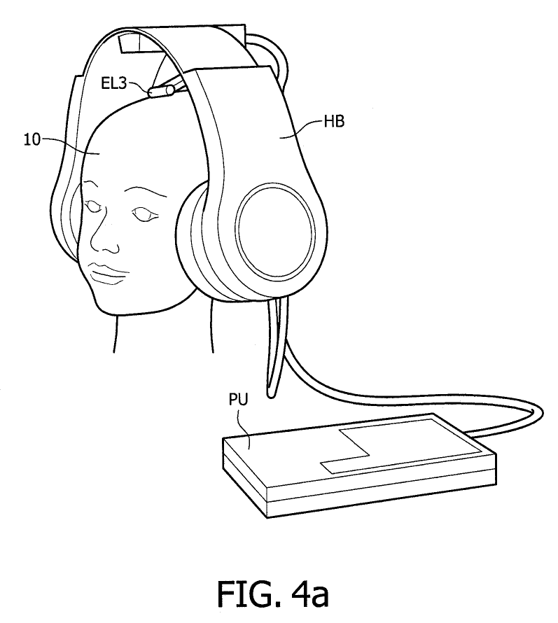

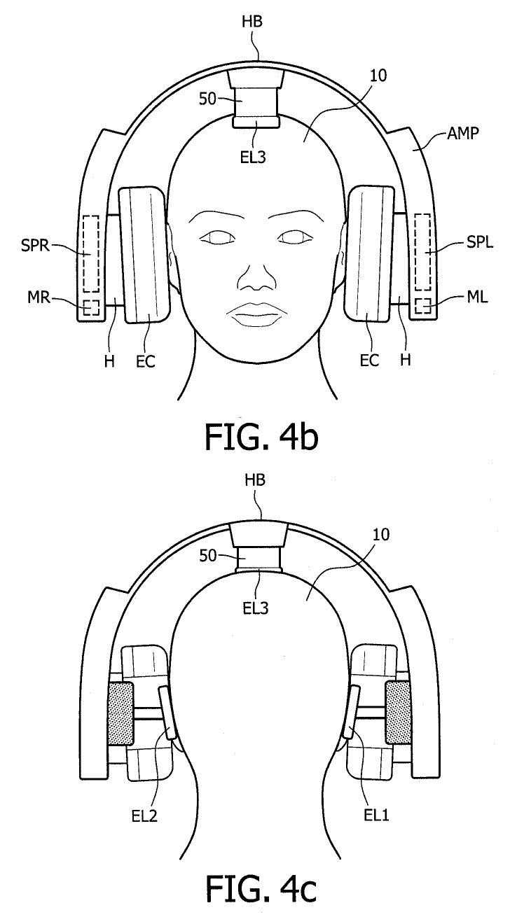

FIG. 4a shows an illustration of a head-set comprising the hearing screening system of FIG. 1 and a processor unit in accordance with an embodiment o the present system. FIG. 4b shows an illustration of a front-side of the head-set of FIG. 4a. FIG. 4c shows an illustration of a rear-side of the head-set of FIG. 4a. The head-set comprises a flexible head-band HB which has ear-caps EC at both ends for receiving an ear of the patient or subject. The head-band HB is designed such that a wide range of head-sizes fit in the head-set. In this embodiment the ear-caps EC are connected to the head-band HB via hinges H which provide for some flexibility in the orientation of the ear-caps EC, but this is not essential. Also, the hinges in accordance with an embodiment of the present system may be designed such that the ear-caps EC can be decoupled for cleaning/sterilizing purposes. At the ends of the head-band HB a speaker SPL for the left ear and a speaker for the right ear SPR is illustratively shown integrated into the head-set. The speakers SPL, SPR are designed for applying auditory stimuli (clicks) to the respective ears. The basic functions of the ear-caps EC are to guide sound emitted by the speakers to the ear channel in an optimal manner, and to prevent environmental sound to reach the ears. At both ends of the head-band HB also microphones ML, MR are illustratively shown integrated to record environmental sound. To that end, in accordance with an embodiment of the present system, these microphones may form a part of a noise-cancellation system which may be coupled to the speakers. On the head-band there is further integrated an integrated amplifier comprising the block as illustrated and discussed in the drawings. In order to make the head-set fit to larger number of patients or subjects the third electrode EL3 is illustratively shown mechanically coupled to the head-band HB via an adjustment device 50, which may be a spring structure in this example, but this is not essential. In accordance with this embodiment, the adjustment device provides a good connection of the third electrode EL3 and the Cz-position on the head of the patient or subject independent of the size of the head, and at the same time prevents the head-set to glide/slip from the head of the patient or subject (double function). The first electrode EL1 is provided at the right mastoid location and the second electrode EL2 is provided at the left mastoid location. In an advantageous embodiment the electrodes may be durable electrodes. In the prior art a lot of electrodes are just thrown away after single use. The use of durable electrodes prevents such waste which is very advantageous for the environment. The electrodes may be formed from stainless steel, silver chloride, or sintered silver chloride, but other suitable materials are not excluded, for example Gold, Platinum, and Rubidium. The electrodes may be mounted to the head-set through an adjustable mounting device, such as springs, or other mountings that may be suitably applied. This embodiment facilitates that the electrodes automatically contact the skin of the subject or patient when the head-set is put on the head. The advantage of using springs is that apart from the mechanical flexibility, also the springs may provide a secure electrical path between the electrodes and the skin (e.g., the electrical current runs through the springs).

In accordance with an embodiment of the present system, the hearing screening system (e.g., a brainstem recorder, a hearing screener headset, etc) may be coupled to a processor unit PU (see FIG. 4a) for screening the hearing of a patient or subject and for rendering a result of testing (e.g., displaying a result, producing an auditory result, etc.).

The brainstem recorder (head-set) in accordance with an embodiment of the present system may be completed as follows. The head-set is placed on the head 10 of a subject or patient, for example a newborn. The two-channel electrophysiological signals are measured and amplified for example, 15,000 times (as discussed earlier) by the two-channel EEG amplifier that is integrated into the head-set. The amplified signals are transmitted to the processor unit. In this example embodiment, the processor unit may include a powerful and energy-saving computer, such as an XSCALE PXA310 (624 MHz clock-frequency) from Toradex. More information on the PXA310 from Toradex is to be found in the datasheets, which are available at toradex.com.

In accordance with an embodiment of the present system a computer program stored in a memory configures the computer (e.g., a processor) to generate a data-array, for example, with 92 clicks per second for the right ear and 90 clicks per second for the left ear. A click in electrical form may be in a form of a square pulse of 100 microseconds in width. The UCB1400-chip on the PXA310-module has a headphone-buffer. Because of this, the computer produces enough power to drive a headphone without additional (external) buffering. Therefore, the stereo-audio line output of the PXA310 may be directly connected to the speakers SPL, SPR in the head-set. The speakers SPL, SPR of the head-set transform the electrical clicks into acoustical stimuli, for example of about 1 msec in length and with a loudness 35 dB nHL (normalized hearing level) being the international accepted loudness level of audible stimuli for hearing screening. The amplified two-channel EEG may be connected to the stereo-audio line input. The stereo-line in signals (EEG) may be analog-digital converted, for example, in synchrony with digital-analog conversion of the stereo-line out signals, such as in exact synchrony. In accordance with this embodiment, the synchronous conversion is a feature for successful detection of evoked responses. This feature has been tested thoroughly. In accordance with an embodiment of the present system, the methods provided by a suitably programmed processor by software to detect responses to the auditory stimuli may be similar to the methods described in the article of John M S and Picton T W, “MASTER: a Windows program for recording multiple auditory steady-state responses”, Computer methods in Biomedicine 2000; 61, 125-150. This document is hereby incorporated by reference in its entirety.

The methods of the present system are particularly suited to be carried out by a computer software program, such program containing modules corresponding to one or more of the individual steps or acts described and/or envisioned by the present system. Such program may of course be embodied in a computer-readable medium, such as an integrated chip, a peripheral device or memory coupled to the processor. As readily appreciated, application data (computer programming software) and other data are received by the processor for configuring the processor to perform operation acts in accordance with the present system. The operation acts include controlling at least one of the auditory stimulus devices to generate an auditory stimulus and to receive responses from one or more electrodes in accordance with the present system. Further, the processor may be suitably programmed to correlate responses locally and/or transmit responses to a remote system for correlation and to cause rendering (e.g., display) of a result (e.g., pass/refer).

An illustrative example of operation of the present system may be provided as follows. In exact synchrony with the audible clicks, the two-channel EEG is accepted. Small periods of 512 ms EEG, so-called epochs, are tested for excessive EEG noise or ambient (audible) noise. If excessive noise is present, this epoch is rejected. If the EEG noise level is below a certain limit, such as about 20-30 μV, and if the ambient noise level inside of the ear-caps is below a certain limit, such as about 15 dB (but this may also be higher), then the epoch is accepted. Sixteen consecutive accepted epochs (16×512=8192 ms of EEG) are put into a second array. A Fast Fourier Transform is applied to this array. If responses to the auditory stimuli are present, they will appear as a sharp peak at exactly the repetition rate (90 Hz or 92 Hz) of the audible stimulus (John et al., 2000, pp 127). An F-test (F-ratio) estimates the probability that a response at a certain frequency (90 Hz or 92 Hz) is significantly above the neighboring frequencies (noise level) (John et al., 2000, pp 127). The significance level can be set at any pre-defined value (e.g. 1%, 0.1% or 0.05%). The result of the F-test (significant/not significant above noise level) is displayed on the computer-unit as a pass/refer. After the first sixteen epochs have been accepted, recordings continue. The next series of epochs is averaged with the first series followed by the computational methods as described above. And so on, till a pass is displayed for the left and the right ear or until a predefined number of accepted epochs (for example, about 512) has been reached.

The computer may provide a user interface, such as through use of a touch screen and/or another display device. Relevant data of the newborns (or other patients or subjects) to be tested may be displayed and controlled through this interface and through suitably programming of a processor of the computer. Progress during hearing-screening (recording time, number of accepted epochs), pass/refer and noise levels may be displayed on the screen.

After a full day of hearing screenings, the results that have been collected in one or more hearing screeners, can be transferred to a central computer. Software running on this central computer may program the processor to calculate statistics of pass/refer rates of a region, state or land. This enables a day by day tracking of the hearing screening results. This software has already been developed in our lab and is in use in Belgium at “Kind en Gezin” and at Depistage Surdité.

The present system in accordance with an embodiment thus provides a hearing screening system for a patient or subject. In the first aspect, the system includes: i) auditory stimulus device SPL, SPR, such as a speaker, for applying an auditory stimulus to at least one ear of the patient or subject; ii) at least three electrodes EL1, EL2, EL3 for electrically connecting to at least three different positions on a head 10 of the patient or subject for measuring respective potential changes in response to the auditory stimulus; iii) two galvanically isolated power supplies; iv) two differential amplifiers DA1, DA2 being arranged for receiving galvanically isolated supply voltages from the power supplies. In the second aspect the system comprises one (or more) EEG channels with one differential amplifier, wherein the differential amplifier DA1 is arranged for amplifying a potential difference between the pair of electrodes for obtaining an amplified EEG signal on an output of the differential amplifier DA1, wherein the differential amplifier DA1 is configured for first amplifying the potential difference with a total differential gain and subsequently taking a difference between respective output potentials of the amplified potential difference. The present system further provides a corresponding hearing screening method. The present system provides a hearing screening system and method which provides for more reliable hearing screening results.

The advantages, features and other aspects of embodiments of the hearing screening system over the prior art may include:
1] The system is for Automated Auditory Brainstem Response (AABR) detection only (not for oto-acoustic emissions);
2] The system can be integrated into a light-weight/portable device comprising:
   a. A computer unit, display of results, input of patient or subject data;
   b. A Brainstem Recorder that is placed on the patients or subjects head;
3] The system in accordance with one embodiment of the present system does not use disposable electrodes (environmental friendly);
4] The system reduces the cost of hearing screening;
5] High sensitivity and high specificity;
6] Two-channel EEG recordings possible with only three electrodes;
7] Low electronic noise level of 0.07 μV (RMS-value) of the two-channel EEG amplifier may be achieved.
8] The EEG amplifier may have a common mode rejection ratio (CMRR) of 140 dB or more. This is achievable because of the 120 dB CMRR of the instrumentation amplifier (See datasheet of the CMRR of the AD8221: gain may be at least 300 times and the relevant frequency band is 3 kHz and below) plus an extra 20 dB because of the first amplifier stage construction with JFET's.
9] Reduced preparation time before the hearing screening starts.
10] Reduced cleaning/removing/storage time after the hearing screening has been finished.
11] Reduced testing time.
12] Reduced energy consumption/use of solar energy may enable hearing screening in a wider/remote area with no access to normal power supply.
13] The hearing screener functionality and the detection algorithm of the auditory evoked responses are independent of age of the person being tested, so not only suitable for newborns.
14] Easy cleaning/sterilization of the ear-caps.
15] Data-transfer of screening devices to a single computer to enable calculation of pass/refer statistics of a region/state/land.
16] Test of the functional integrity of the hearing screener at startup/connection to computer.
17] Test functional integrity/diagnose functional problem of the hearing screener from a distance is possible.

The system and method described herein address problems in prior art systems. The present system may be applied in various application areas. For example, the present system may be applied in hearing screening systems and methods, but also systems adapted to make audiograms. The present system may be applied in hearing diagnosing systems and methods, the present system may be applied in cerebral function monitoring, the present system may be applied in heart function monitoring.

Various variations of the hearing screening system and method in accordance with the present system are possible and do not depart from the scope of the present system as claimed. These variations for example relate to integration of the computer unit, of the display, input/output terminals and input/output of patient or subject data into the head-set (Brainstem Recorder), wireless transmission of data between the Brainstem Recorder and the computer unit, adding one or more extra EEG channels to the Brainstem Recorder, control the hearing screener unit from a distance (e.g. by internet) before, during or after hearing screening.

Finally, the above discussion is intended to be merely illustrative of the present system and should not be construed as limiting the appended claims to any particular embodiment or group of embodiments. It should be noted that the above-mentioned embodiments illustrate rather than limit the present system, and that those skilled in the art will be able to design many alternative embodiments without departing from the scope of the appended claims. In the claims, any reference signs placed between parentheses shall not be construed as limiting the claim. The section headings included herein are intended to facilitate a review but are not intended to limit the scope of the present system. Accordingly, the specification and drawings are to be regarded in an illustrative manner and are not intended to limit the scope of the appended claims. Any reference signs in the claims are provided to facilitate a review and do not limit the claim scope. Use of the verb “comprise” and its conjugations does not exclude the presence of elements or steps other than those stated in a claim. The article “a” or “an” preceding an element does not exclude the presence of a plurality of such elements. The present system may be implemented by means of hardware comprising several distinct elements, and by means of a suitably programmed computer. Any of the disclosed elements may be comprised of hardware portions (e.g., including discrete and integrated electronic circuitry), software portions (e.g., computer programming), and any combination thereof. In the device claim enumerating several means, several of these means may be embodied by one and the same item of hardware. The mere fact that certain measures are recited in mutually different dependent claims does not indicate that a combination of these measures cannot be used to advantage. The term “plurality of” an element includes two or more of the claimed element, and does not imply any particular range of number of elements; that is, a plurality of elements may be as few as two elements, and may include an immeasurable number of elements. Throughout the Figures, similar or corresponding features are indicated by same reference numerals or labels.

1. A hearing screening system for a subject or patient, the system comprising:
auditory stimulus device configured to apply an auditory stimulus to at least one ear of the subject or patient;
at least three electrodes for electrically connecting to at least three different positions on a head of the subject or patient for measuring respective potential changes in response to the auditory stimulus;
a first power supply having first supply terminals for supplying a first supply voltage;
a second power supply having second supply terminals for supplying a second supply voltage;
a first differential amplifier being arranged for receiving the first supply voltage, the first differential amplifier having a first pair of inputs coupled to a first pair of electrodes selected from the at least three electrodes, the first differential amplifier being arranged for amplifying a first potential difference between the first pair selected from the electrodes for obtaining a first electroencephalographic (EEG) signal on a first output of the first differential amplifier, and
a second differential amplifier being arranged for receiving the second supply voltage, the second differential amplifier having a second pair of inputs coupled to a second pair of electrodes that is different than the first pair of electrodes and that is selected from the at least three electrodes, the second differential amplifier being arranged for amplifying a second potential difference between the second pair selected from the electrodes for obtaining a second EEG signal on a second output of the second differential amplifier;
wherein the first supply voltage and the second supply voltage are galvanically isolated from each other.

2. The hearing screening system as claimed in claim 1, wherein at least one of the first supply terminals is coupled to a first specific one of the electrodes, and wherein at least one of the second supply terminals is coupled to a second specific one of the electrodes, wherein the second specific one is different from the first specific one.

3. The hearing screening as claimed in claim 2, wherein the first supply terminals further comprise a first intermediate supply terminal for supplying a first intermediate supply potential that is located between respective potentials of the first supply voltage, and wherein the second supply terminals further comprise a second intermediate supply terminal for supplying a second intermediate supply potential that is located between respective potentials of the second supply voltage, wherein the first intermediate supply terminal is coupled to the first specific one of the electrodes, and wherein the second intermediate supply terminal is coupled to the second specific one of the electrodes.

4. The hearing screening system as claimed in claim 2, wherein the at least three electrodes comprise a first electrode, a second electrode and a third electrode, respectively, wherein the first pair of inputs is coupled to the first electrode and the third electrode, and wherein the second pair of inputs is coupled to the second electrode and the third electrode.

5. The hearing screening system as claimed in claim 4, wherein
the first specific one of the electrodes that is coupled to the at least one of the first supply terminals, is the second electrode, and wherein the second specific one of the electrodes that is coupled to the at least one of the second supply terminals, is the first electrode.

6. The hearing screening system as claimed in claim 1, wherein each respective one of the first pair of inputs is provided with a first coupling, for simultaneous DC-decoupling from and AC-coupling to a respective one of the first pair of electrodes, and wherein each respective one of the second pair of inputs is provided with second coupling for simultaneous DC-decoupling from and AC-coupling to a respective one of the second pair of electrodes.

7. The hearing screening system as claimed in claim 6, wherein respective coupling comprise a respective coupling capacitor per input of the respective differential amplifiers, each respective coupling capacitor being connected between a respective electrode and a respective input.

8. The hearing screening system as claimed in claim 1, wherein the differential amplifiers are configured for a total differential gain between 1000 and 200000.

9. The hearing screening system as claimed in claim 8, wherein the differential amplifiers are configured for first amplifying the respective potential differences with the total differential gain and subsequently taking a difference between respective output potentials of the amplified potential differences.

10. The hearing screening system as claimed in claim 1, further comprising two isolation amplifiers and a third power supply, wherein each respective one of the differential amplifiers is coupled to a respective one of the isolation amplifiers, wherein the isolation amplifiers each comprise a galvanic barrier between a respective input side and a respective output side of the isolation amplifiers, wherein the respective output sides are each coupled to the third power supply for setting a DC level of respective outputs of the isolation amplifiers to obtain a first output channel and a second output channel respectively.

11. The hearing screening system as claimed in claim 1, wherein the system is integrated into a head-set for mounting on a head of a subject or patient.

12. The hearing screening system as claimed in claim 11, wherein the head-set is designed such that, in operational use, a position of the first electrode coincides with the right-mastoid position, a position of the second electrode coincides with the left-mastoid position, and a position of the third electrode coincides with the Cz-position, respectively.

13. The hearing screening system as claimed in claim 1, wherein the electrodes are durable electrodes.

14. The hearing screening system as claimed in claim 1, wherein the auditory stimulus device is configured for applying the auditory stimulus on both ears simultaneously at different repetition rates.

15. The hearing screening system as claimed in claim 1, further comprising a processor unit connected to the auditory stimulus device for controlling the application of the auditory stimulus, the processor unit being further configured for receiving, collecting, and processing data from the differential amplifier to obtain quantitative data about the hearing ability of the subject or patient.

16. The hearing screening system as claimed in claim 15, wherein the processor unit is arranged for calculating a third EEG channel by subtracting the first channel from the second channel.

17. A hearing screening system for a subject or patient, the system comprising:
auditory stimulus device configured to apply an auditory stimulus to at least one ear of the subject or patient;
at least two electrodes for electrically connecting to at least two different positions on a head of the subject or patient for measuring respective potential changes in response to the auditory stimulus;
a differential amplifier having a pair of inputs coupled to a pair selected from the electrodes, the differential amplifier being arranged for amplifying a potential difference between the pair of electrodes for obtaining an amplified electroencephalographic (EEG) signal on an output of the differential amplifier, wherein the differential amplifier is configured for a total differential gain between 1000 and 200000, and wherein the differential amplifier is configured for first amplifying the potential difference with the total differential gain and subsequently taking a difference between respective output potentials of the amplified potential difference.

18. A hearing screening method for a subject or patient, the method comprising acts of:
providing at least two electrodes on at least two different positions on a head of the subject or patient;
applying an auditory stimulus to at least one ear of the subject or patient;
measuring potential changes in response to the auditory stimulus with the at least two electrodes, and
amplifying a potential difference between the electrodes with a total differential gain between 1000 and 200000 for obtaining an amplified electroencephalographic (EEG) signal, and
taking a difference between respective output potentials of the amplified potential difference.
